(19) United States
(12) Patent Application Publication
(10) Pub. No.: US 2025/0161632 A1
(43) Pub. Date: May 22, 2025

(54) MEDICAL APPARATUS WITH WIRE ANCHORS AND METHOD OF USE THEREOF

(71) Applicant: Canon U.S.A., Inc., Melville, NY (US)

(72) Inventor: Benedict Shia, Needham, MA (US)

(21) Appl. No.: 18/836,989

(22) PCT Filed: Feb. 8, 2023
(86) PCT No.: PCT/US23/12630

§ 371 Date: Aug. 8, 2024

Related U.S. Application Data
(60) Provisional application No. 63/308,432, filed on Feb. 9, 2022.

(51) Int. Cl. A61M 25/01; A61B 1/005; A61M 25/00

(52) U.S. Cl. CPC A61M 25/0108; A61M 25/0009; A61B 1/0057; A61M 2025/015

(57) ABSTRACT

An articulated medical device having a hollow core, wherein the device is capable of maneuvering through cavities to reach a target with minimal invasiveness, and once the medical device has reached the target, allowing a medical tool to be guided through the hollow cavity for facilitating medical procedures, including endoscopes, cameras, and catheters, at the target.

### CROSS REFERENCE TO RELATED PATENT APPLICATIONS

This application claims priority from U.S. Provisional Patent Application No. 63/308,432, filed on Feb. 9, 2022, in the United States Patent and Trademark Office, the disclosure of which is incorporated by reference herein, in its entirety.

### FIELD OF THE DISCLOSURE

The present disclosure relates generally to apparatus and methods for medical application. More particularly, the subject disclosure is directed to an articulated medical device having a hollow cavity, wherein the device is capable of maneuvering within a patient, and allowing a medical tool to be guided through the hollow cavity for medical procedures, including endoscopes, cameras, and catheters.

### BACKGROUND OF THE DISCLOSURE

Bendable medical instruments such as endoscopic surgical instruments and catheters are well known and continue to gain acceptance in the medical field. The bendable medical instrument generally includes a flexible body commonly referred to as a sleeves or sheaths. One or more tool channels extend along (typically inside) the flexible body to allow access to a target located at a distal end of the body.

The instrument is intended to provide flexible access within a patient, with at least one curve or more leading to the intended target, while retaining torsional and longitudinal rigidity so that a physician can control the tool located at the distal end of the medical instrument by maneuvering the proximal end of the medical instrument.

Recently, to enhance maneuverability of the distal end of the instrument, robotized instruments that control distal portions have emerged. In those robotized instruments, to create curves locally at the distal portion by robotics, different techniques have been disclosed.

By way of example, United States patent publication number 2016/0067450, provides multiple conduits to retain the shape of the proximal part, while the driving tendons are bending the distal part in the medical instruments. The multiple conduits would be controlled selectively in a binary way by constraining or unconstraining the proximal ends of the conduits. By selecting the constrained conduits, the bendable medical device can change the length of bending distal segment by changing the stiffness of the bendable medical device based on the area where the conduits deploy.

However, there remains a need in the industry to further refine and advance bendable medical devices to minimize the outer size (diameter) of the bendable medical instrument, and to maximize the size (diameter) of the tool channel allowing for larger/more effective tools.

In order to maximize the bending output of the medical instrument. The wire needs to have some anchoring mechanism to secure it to the plastic ring. The strength of the bond between the anchor and the wire, as well as the strength of the bond between the anchor to the plastic ring, will determine the limits of how hard the wires can be pulled and pushed, determining how much deflection can be effected.

### SUMMARY

Thus, to address such exemplary needs in the industry, the presently disclosed apparatus teaches a medical apparatus comprising: a bendable body comprising: a hollow cavity extending the length of the bendable body; and a wall formed about the hollow cavity; at least one control wire slideably situated in the wall; and an anchor configured at a distal end of the at least one control wire, wherein, the anchor is affixed within the wall.

In other embodiments, the anchor is affixed to the control wire by crimping. Alternatively, the anchor is affixed to the control wire by welding.

Furthermore, the wherein the anchor is affixed to the control wire by crimping and welding.

In other embodiment, the anchor further comprises a feature creating additional surface area on an outside surface of the anchor for increasing an attachment strength of the anchor to the wall. In addition, the feature creating additional surface area on the outside surface of the anchor is selected from the groups consisting of a notch, a hole, a fissure, a loop, and combinations thereof.

In other contemplated embodiment, the at least one control wire includes a plurality of anchors, wherein the position of each anchor is different along the axial direction of the bendable body.

Furthermore, the apparatus may further comprise a driving unit in communication with the at least one control wire, configured to actuate the at least one control wire in the wall.

In additional embodiment, the at least one control wire further comprises an outer wire and an inner wire, wherein the inner wire is slideably nested within the outer wire. Furthermore, the outer wire has an anchor for affixing the outer wire to the wall and the inner wire has an anchor for affixing the inner wire to the wall, wherein the position of the anchor for the outer wire and the inner wires are different along the axial direction of the bendable body.

In yet another embodiment, the control wire and anchor comprise of a radio opaque material.

Additionally, the apparatus may further comprise a functional probe selected from the group consisting of a position tracking sensor, a shape sensor, an endoscopic imaging probe, derivatives thereof, and combinations therefrom.

Furthermore, the affixing the anchor to the wall comprises heating the wall and anchor to create a thermal fusion.

In yet another scenario, the wall comprises at least two wire guides, wherein the wire guides are spaced a distance from one another.

The subject innovation also teaches a medical apparatus prepared by a process comprising the steps of: providing a bendable body comprising: a hollow cavity extending the length of the bendable body; a wall formed about the hollow cavity; at least one control wire slideably situated in the wall; and an anchor configured at a distal end of the at least one control wire; heating the anchor configured in the wall to create a fusion between the wall and anchor; and cooling the anchor configured in the wall to set the fusion.

Furthermore, the subject innovation discloses, a method for treating a subject, comprising: providing a medical apparatus comprising: a bendable body having a hollow cavity extending the length of the bendable body, and a wall formed about the hollow cavity; at least one control wire slideably situated in the wall; and an anchor configured at a distal end of the at least one control wire wherein the anchor is affixed to the wall; advancing the medical apparatus into a subject; bending the medical apparatus to accommodate obstacles in the subject; and treating the subject once the medical apparatus advances to a desired target in the subject.

The subject apparatus may be used in a variety of applications and by a variety of methods, including the steps of: providing a bendable body comprising: a hollow cavity extending the length of the bendable body; a wall formed about the hollow cavity; at least one control wire slideably situated in the wall; and an anchor configured at a distal end of the at least one control wire; heating the anchor configured in the wall to create a fusion between the wall and anchor; and cooling the anchor configured in the wall to set the fusion.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 is a block diagram of an exemplary bendable medical device incorporating various ancillary components, according to one or more embodiment of the subject apparatus, method or system.

FIG. 2A depicts a perspective close-up view of an exemplary bendable medical device, according to one or more embodiment of the subject apparatus, method or system.

FIG. 2B depicts a perspective view of an exemplary bendable medical device, according to one or more embodiment of the subject apparatus, method or system.

FIG. 2C depicts a perspective cross-view view of an exemplary bendable medical device, according to one or more embodiment of the subject apparatus, method or system.

FIG. 3 provides a side perspective view of an anchor and control wire in an exemplary bendable medical device, according to one or more embodiment of the subject apparatus, method or system.

FIG. 4 is a side perspective view of an anchor and control wire in an exemplary bendable medical device, according to one or more embodiment of the subject apparatus, method or system.

FIG. 5 provides a side perspective view of an exemplary bendable medical device anchor and control wire, according to one or more embodiment of the subject apparatus, method or system.

FIG. 6 depicts a side perspective view of an anchor and control wire in an exemplary bendable medical device, according to one or more embodiment of the subject apparatus, method or system.

FIG. 7 illustrates a side perspective view of an anchor and control wire in an exemplary bendable medical device, according to one or more embodiment of the subject apparatus, method or system.

FIG. 8 provides a side perspective view of an anchor and control wire in an exemplary bendable medical device, according to one or more embodiment of the subject apparatus, method or system.

FIG. 9 is a side perspective view of an anchor and control wire in an exemplary bendable medical device, according to one or more embodiment of the subject apparatus, method or system.

FIG. 10 is an illustration of a side perspective view of an anchor and control wire in an exemplary bendable medical device, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

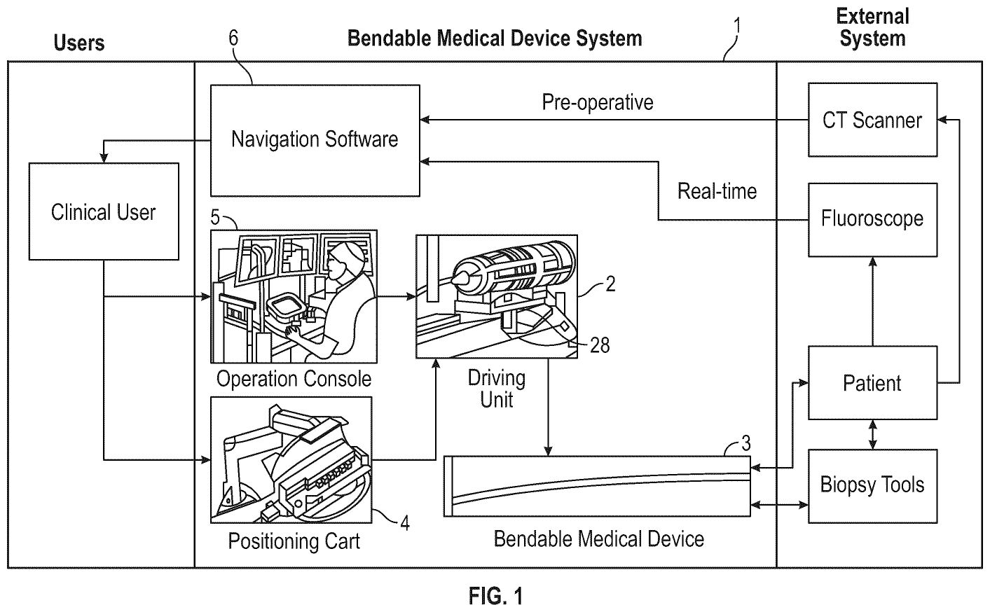

FIG. 1 is a system block diagram of an exemplary bendable medical device system 1 incorporating various ancillary components intended to amass a complete medical system. The bendable medical device system 1 comprises an actuator or driving unit 2 (also referred to herein as a ‘driver’) for driving the wires, and having a base stage 28, a bendable medical device 3, a positioning cart 4, an operation console 5, having push-button, thumbstick, and/or joystick operational console 5, and navigation software 6. The operation console 5 includes a user input device such as a push-button, thumbstick, and/or joystick. The operation console 5 also includes a processor for processing input from the user as well as input from sensors, etc. The processor further processes mapping between the input and the movement of the driver and movement of the base.

The navigation software 6 and the driving unit 2 are communicatively coupled via a bus to transmit/receive data between each other. Moreover, the navigation software 6 is connected and may communicate with a CT scanner, a fluoroscope and an image server (not in Figure), which are ancillary components of the bendable medical device system 1. The image server may include, but is not limited to, a DICOM™ server connected to a medical imaging device including but not limited to a CT and/or MRI scanner and a fluoroscope. The navigation software 6 processes data provided by the driving unit 2 and data provided by images stored on the image server, and/or images from the CT scanner and the fluoroscope in order to display images onto the image display.

The images from the CT scanner may be pre-operatively provided to navigation software 6. With navigation software, a clinical user creates an anatomical computer model from the images. In this particular embodiment, the anatomy is that of a lung with associated airways. From the chest images of the CT scanner, the clinical user can segment the lung airways for clinical treatments, such as biopsy. After generating the lung airway map, the user can also create plan to access the lesion for the biopsy. The plan includes the airways to insert and maneuver the bendable medical device 3 leading to the intended target, which in this example is a lesion.

The driving unit 2 comprises actuators and a control circuitry. The control circuitry is communicatively-coupled with operation console 5. The driving unit 2 is connected to the bendable medical device 3 so that the actuators in the driving unit 2 operate the bendable medical device 3. Therefore, a clinical user can control the bendable medical device 3 via the driving unit 2. The driving unit 2 is also physically connected to a positioning cart 4. The positioning cart 4 includes a positioning arm, and locates the driving unit 2 and the bendable medical device 3 in the intended position with respect to the target/patient. The clinical user can insert, maneuver and retreat the bendable medical device 3 to perform medical procedures, here a biopsy in the lungs of the patient.

The bendable medical device 3 can be navigated to the lesion in the airways based on the plan by the clinical user's operation. The bendable medical device 3 includes a hollow cavity for various tools (e.g. a biopsy tool). The bendable medical device 3 can guide the tool to the lesion of the patient. In one example, the clinical user can take a biopsy sample from the lesion with a biopsy tool.

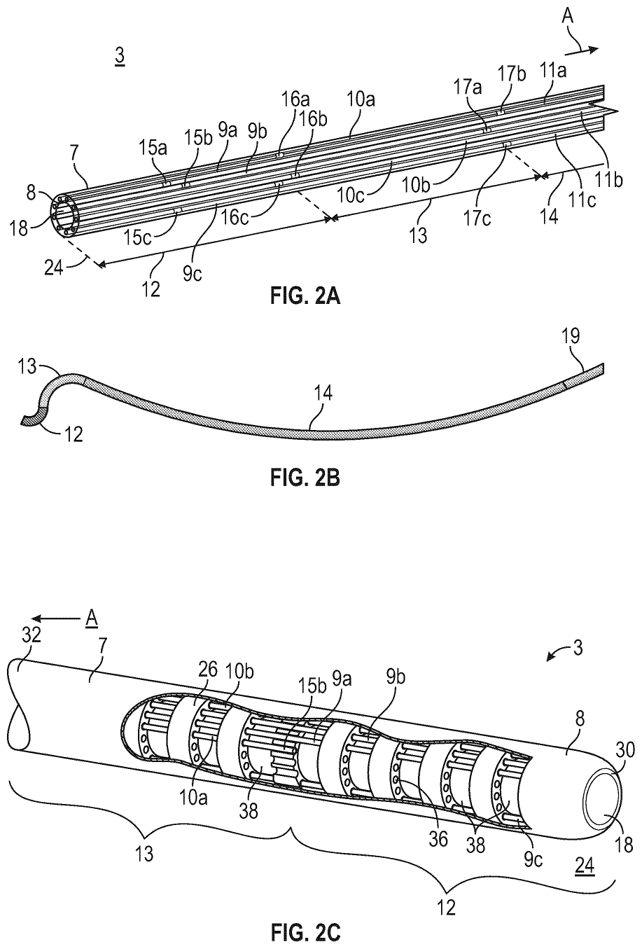

FIGS. 2A, 2B and 2C are schematic drawings of the bendable medical device 3, with FIG. 2A detailing a perspective close-up view of the bendable medical device 3, and FIG. 2B depicting a schematic drawing to explain the bendable segments of the bendable medical device 3. The bendable medical device 3 has a distal end 24 and a proximal end (in direction of arrow A), and comprises a proximal segment 19 and three bendable segments, which are the first, second, and third bendable segments 12, 13, 14, respectively (see FIG. 2B).

The bendable segments 12, 13, 14, can independently bend and can form a shape with three independent curvatures, as seen in FIG. 2B. The bendable medical device 3 includes a bendable body 7 with an inner diameter 30 and an outer diameter 32, which creates the cylindrical wall 8 of the bendable body 7, wherein the inner diameter 30 establishes a tool channel 18 (see FIG. 2C). The wall 8 may house several lumens 26 intended to house one or more control wires (detailed below), wherein the lumens 26 are spaced a distance from one another creating at least one cavity 38 that greatly enhancing the bendable angle of the bendable segment 12, 13 and 14. The tool channel 18 is configured to extend the length of the bendable body 7, wherein the proximal part 19 of the bendable body 7 provides access to clinical users for inserting/retreating a medical tool. For example, a clinical user can insert and retrieve a biopsy tool trough the tool channel 18 to the distal end 24 of the bendable medical device 3. This may be accomplished after the bendable device 3 is inserted into the subject, or in unison with insertion/retreating the bendable device 3.

The bendable body 7 includes a set of first control wires 9a, 9b, 9c, a second set of control wires 10a, 10b, 10c, and a third set of control wires 11a, 11b, 11c (See FIG. 2A). The wall 8 houses the control wires 9a-11c through the lumens 34, which are configured along longitudinal direction of the bendable body 7. The lumens 34 have fissures 36 for each wire, thus allowing for slideable movement of the control wires 9a-11c along an axial direction of the bendable body. The control wires 9a-11c may be terminated at the distal end of each bendable segment 12, 13 and 14, to form the three bendable groups, each with three wires each (a, b, c). The first control wires 9a, 9b, 9c are terminated at the distal end of the first bendable segment 12 with anchoring segments 15a, 15b, 15c, and are configured apart from each other by approximately 120 degrees within the wall 8. The first control wires 9a, 9b, 9c are connected to the driving unit 2 at the proximal end of the wires 9a, 9b, 9c. The driving unit 2 induces pushing or pulling forces to move the control wires 9a, 9b, 9c by actuating those wires, and bending the bendable body 7 from the distal end 24.

Similarly, the second set of control wires 10a, 10b, 10c are terminated at the distal end of the second bendable segment 13, using the anchoring segments 16a, 16b, 16c, and are connected to the driving unit 2 at the proximal end. The second set of control wires 10a, 10b, 10c are also housed in the wall 8. The second set of control wires 10a, 10b, 10c can bend the bendable body 7 from the distal end of the second bendable segment 13.

In the same way, the third set of control wires 11a, 11b, 11c are also configured to bend the bendable body 7 at the third bendable segment 14, once again by inducing pushing or pulling, and by anchoring segment 17a, 17b, 17c, respectively, which are actuated at the distal end 24 of the control wires 11a, 11b, 10c by the driving unit 2.

Accordingly, by pushing and pulling the set of control wires 9, 10, 11, the first, the second and the third bendable segments 12, 13, 14, respectively, individually bend the bendable medical device 3, in three dimensions.

The subject bendable medical device 3 incorporates control wires 9, 10, 11, that can be fixed to the bendable body 7 by using minimal space in the bendable body wall 8. Because the anchoring segment 15-17 is localized within the individual lumens 34, the bendable medical device 3 with the control wires 9, 10, 11, can be effectively miniaturized, especially when using multiple control wires 9, 10, 11. Additionally, the control wires 9, 10, 11, can be fully contained within the bendable body 7 wall 8, not needing to be outside the outer diameter 32 or inside the inner diameter 30; thus not impinging on the tool channel 18 or unnecessarily increasing the overall size of the medical device 3. By embedding the anchor segments 15-17 in the wall 8 of the bendable body 7, the control wires 9, 10, 11, can transmit pushing force, torque as well as pulling force to the bendable body 7. Therefore, the bendable medical device 3 can reduce the number of control wires 9, 10, 11, or force load per the control wire 9, 10, 11, to achieve the target bending maneuver in comparison to the conventional tendon-driven system with pulling forces.

Moreover, because the anchoring segment 15-17 does not need additional abutment parts that cover the cross section of the bendable body 7, the bendable medical device 3 can reduce the number of jointing points between the bendable body 7 and those additional abutment parts at the position of the anchoring segment 15-17. Therefore, the subject bendable medical device 3 can improve continuity of flexibility with reduced influence of motion control and reduce risks of failure in those jointing points. Also, the bendable medical device 3 can have a continuous smooth surface on the outer diameter 32 and the inner diameter 30 of the bendable body 7 to avoid risk of trauma to the patient anatomy and improved tool advancement/retraction in the tool channel 18.

Furthermore, the control wire 9, 10, 11, can be fixed to the bendable body at a wide variety of positions along the length of the bendable body 7, allowing the bendable medical device 3 to be configured to have multiple bending segments, especially a distal bending segment manipulated independently from the proximal part of the bendable body to provide improved flexible access to the intended treatment area of the patient.

Our attention will now turn to the anchoring segments 15-17, and more specifically, to various anchors to be implemented in the anchoring segments 15-17.

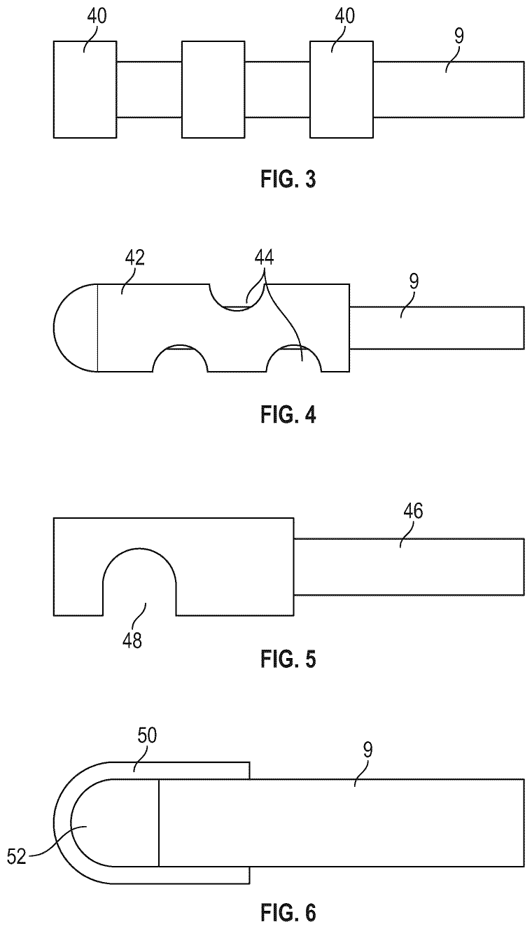

FIG. 3 provides the use of rings 40 could be welded to the control wire 9. The rings would provide a more regular and predictable location for welds, and also more area for the welds, resulting in greater tensile strength. The rings 40 would be best suited to be made from the same material as the control wire 9.

Another concept, seen in FIG. 4, would be to place a tube 42 onto the end of the control wire 9. The tube 42 would ideally be made out of the same material as the control wire 9, so the two can be welded together at the distal end. In order to provide anchoring in the plastic, notches or holes 44 can be cut into the tube 42, or the tube 42 could be crimped or knurled to provide texture.

Another tube concept depicted in FIG. 5 would be to attach a tube 46 with a notch or hole 48 to the end of the control wire 9, with the notch or hole 48 would be past the end of the control wire 9. The notch or hole 48 would provide purchase for the plastic to anchor the tube 46 in the plastic.

Yet another concept depocted in FIG. 6 would be to connect a ribbon wire 50 to the end of the control wire 9 to form a loop 52. The loop 52 would provide purchase for the plastic to anchor the ribbon wire 50 in the plastic.

These concepts are all based on the control wire 9 and anchor being made of the same material. If the anchor is a different material than the control wire 9, welding is no longer as good an option to connect the two components. A mechanical attachment, such as crimping is more likely to provide an acceptable bond.

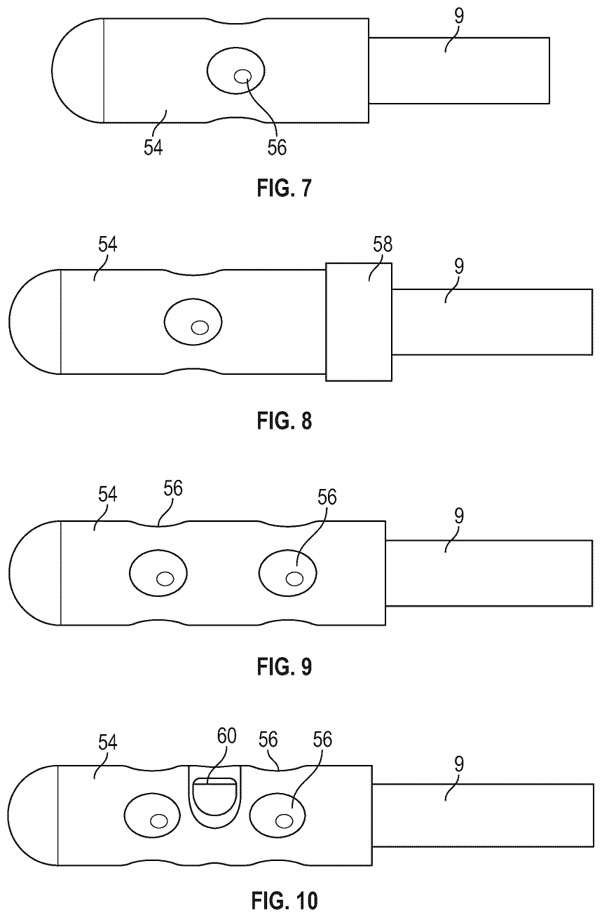

The concepts shown in FIGS. 7-10 provide a tube 54 that is crimped onto the control wire 9. The end of the control wire 9 is ball welded to form a mechanical stop. The ball weld keeps the control wire 9 from being pulled out of the tube 54, while the crimp 56 does the same, but also keeps the control wire 9 from being pushed out of the tube. The crimp 56 provide purchase for the plastic to anchor the assembly in the plastic.

Another configuration of the above concept is to attach a ring 58 of the same material as the tube 54, but at a larger diameter, as seen in FIG. 8. This will provide extra purchase in the plastic.

Further configuration could include multiple crimps 56, as seen in FIG. 9. The additional crimp(s) 56 provide better mechanical attachment between the tube 54 and the control wire 9, and will provide additional texture for purchase in the plastic. In addition, notches or holes 60 may be cut into the tube 54 for added integrity (See FIG. 10).

As can be appreciated, the subject innovation provides increased attachment strength between the anchor and the control wire, and between the anchor and the plastic ring, allowing for greater forces to be applied to the ring to effect deflection. It is also important that the increased attachment strength affects the actuation when the control wire is pushed as well as pulled. This is especially beneficial when multiple control wires are used to effect deflection, as opposing control wires could be pulled and pushed to maximize deflection and increase degrees of circulation for movement into more obscure fissures of the anatomy.

1. A medical apparatus comprising:
a bendable body comprising:
a hollow cavity extending the length of the bendable body; and
a wall formed about the hollow cavity;
at least one control wire slideably situated in the wall; and
an anchor configured on the at least one control wire,
wherein, the anchor is affixed within the wall.

2. The apparatus of claim 1, wherein the anchor is affixed to the control wire by crimping.

3. The apparatus of claim 1, wherein the anchor is affixed to the control wire by welding.

4. The apparatus of claim 1, wherein the anchor is affixed to the control wire by crimping and welding.

5. The apparatus of claim 1, wherein the anchor further comprises a feature creating additional surface area on an outside surface of the anchor for increasing an attachment strength of the anchor to the wall.

6. The apparatus of claim 5, wherein the feature creating additional surface area on the outside surface of the anchor is selected from the groups consisting of a notch, a hole, a fissure, a loop, and combinations thereof.

7. The apparatus of claim 1, wherein the at least one control wire includes a plurality of anchors, wherein the position of each anchor is different along the axial direction of the bendable body.

8. The apparatus of claim 1, further comprising a driving unit in communication with the at least one control wire, configured to actuate the at least one control wire in the wall.

9. The apparatus of claim 1, wherein the at least one control wire further comprises an outer wire and an inner wire, wherein the inner wire is slideably nested within the outer wire.

10. The apparatus of claim 9, wherein the outer wire has an anchor for affixing the outer wire to the wall and the inner wire has an anchor for affixing the inner wire to the wall, wherein the position of the anchor for the outer wire and the inner wires are different along the axial direction of the bendable body.

11. The apparatus of claim 1, wherein the control wire and anchor comprise of a radio opaque material.

12. The apparatus of claim 1, further comprising a functional probe selected from the group consisting of a position tracking sensor, a shape sensor, an endoscopic imaging probe, derivatives thereof, and combinations therefrom.

13. The apparatus of claim 1, wherein affixing the anchor to the wall comprises heating the wall and anchor to create a thermal fusion.

14. The apparatus of claim 1, wherein the wall comprises at least two wire guides, wherein the wire guides are spaced a distance from one another.

15. A medical apparatus prepared by a process comprising the steps of:
providing a bendable body comprising:
a hollow cavity extending the length of the bendable body;
a wall formed about the hollow cavity;
at least one control wire slideably situated in the wall; and
an anchor configured at a distal end of the at least one control wire;
heating the anchor configured in the wall to create a fusion between the wall and anchor; and
cooling the anchor configured in the wall to set the fusion.

16. The apparatus of claim 15, wherein the anchor is affixed to the control wire by crimping.

17. The apparatus of claim 15, wherein the anchor is affixed to the control wire by welding.

18. The apparatus of claim 15, wherein the anchor is affixed to the control wire by crimping and welding.

19. The apparatus of claim 15, wherein the anchor further comprises a feature creating additional surface area on an outside surface of the anchor for increasing an attachment strength of the anchor to the wall.

20. A method for treating a subject, comprising:
providing a medical apparatus comprising:
a bendable body having a hollow cavity extending the length of the bendable body, and a wall formed about the hollow cavity;
at least one control wire slideably situated in the wall; and
an anchor configured at a distal end of the at least one control wire
wherein the anchor is affixed to the wall;
advancing the medical apparatus into a subject;
bending the medical apparatus to accommodate obstacles in the subject; and
treating the subject once the medical apparatus advances to a desired target in the subject.
